(19) United States
(12) Patent Application Publication
(10) Pub. No.: US 2007/0141126 A1
(43) Pub. Date: Jun. 21, 2007

(54) Germicidal surface-covering assembly

(76) Inventors: Tammy McFall Hudson, Suwanee, GA (US); David William Koenig, Menasha, WI (US); Phillip Andrew Schorr, Atlanta, GA (US); Douglas Robert Hoffman, Greenville, WI (US); Sara Ann Carney, De Pere, WI (US); Lisa M. Kroll, Appleton, WI (US)

Correspondence Address: KIMBERLY-CLARK WORLDWIDE, INC., 401 NORTH LAKE STREET, NEENAH, WI, 54956, US

(21) Appl. No.: 11/313,456

(22) Filed: Dec. 21, 2005

(51) Int. Cl. A61K 9/70; B32B 27/04

(52) U.S. Cl. 424/443; 442/123

(57) ABSTRACT

A germicidal surface-covering assembly that includes at least two different donnable or drapable garments. Each garment defines at least one treated surface that is susceptible to pathogen contamination in a physical contamination event when used as intended in an environment subject to contamination (e.g., a clinical environment, a laboratory or a workplace). Each treated surface is adapted to provide a time-dependent reduction in the number of pathogens available at that treated surface after a physical contamination event, such that at least a predetermined time after a physical contamination event at a first location on a first treated surface of a first garment, a first physical contact between the first location on the first treated surface and a second location on a second treated surface of a second garment results fewer viable pathogens on the second treated surface as compared to an untreated control. Moreover, at least a predetermined time after the first physical contact, a second physical contact between the second location on the second treated surface and a third location on a third treated surface of a third garment results in fewer viable pathogens on the third treated surface as compared to an untreated control.

### FIELD OF INVENTION

The present invention relates to drapable or donnable articles. In particular, the invention relates to articles for controlling the spread of pathogens and infectious diseases.

### BACKGROUND

In recent years, the prevalence of nosocomial infections has had serious implications for both patients and healthcare workers. Nosocomial infections are those that originate or occur in a hospital, long-term care facility, or other health care setting, and are sometimes referred to as “hospital associated infections” or HAI. In general nosocomial infections are more serious and dangerous than external, community-acquired infections because the pathogens in hospitals are more virulent and resistant to typical antibiotics. Nosocomial infections are responsible for about 90,000 deaths in the United States per year. About 5% to 10% of American hospital patients (about 2 million per year) develop a clinically significant nosocomial infection. These HAIs are usually related to a procedure or treatment used to diagnose or treat the patient's illness or injury and may be spread by indirect, inadvertent contact.

Infection control has been a formal discipline in the United States since the 1950s, due to the spread of staphylococcal infections in hospitals. Because there is both the risk of health care providers acquiring infections themselves, and of them passing infections on to patients, the Centers for Disease Control and Prevention have established guidelines for infection control procedures. In addition to hospitals, infection control is important in nursing homes, clinics, physician offices, child care centers, and restaurants, as well as in the home. The purpose of infection control in hospital and clinical environments is to reduce the occurrence of infectious diseases. These diseases are usually caused by bacteria or viruses and can be spread by human to human contact, animal to human contact, human contact with an infected surface, airborne transmission, and, finally, by such common vehicles as food or water. The use of medical devices such as gloves, gowns, and masks as barriers to pathogens is already well appreciated by infection control practitioners. It is apparent by the increase in antibiotic resistance and the persistence of HAIs, however, that these practices alone are not enough.

Hospitals and other healthcare facilities have developed extensive infection control programs to prevent nosocomial infections. Even though hospital infection control programs and a more conscientious effort on the part of healthcare workers to take proper precautions when caring for patients can prevent some of these infections, a significant number of infections still occur. Therefore, the current procedures are not sufficient. Despite enforcement of precautionary measures (e.g. washing hands, wearing gloves, face mask and cover gowns), contact transfer is still a fundamental cause of HAIs. That is, individuals who contact pathogen-contaminated surface such as table tops, bed rails, hands, clothing and/or medical instruments, can still transfer the pathogens from one surface to another immediately or within a short time after initial contact.

To improve this situation, a standard device or article can be enhanced for infection control by addition of actives that can kill pathogens when they come in contact with the article or can bind the pathogen such that dispersal is not possible. Using a single enhanced device or article alone, however, still leaves other untreated devices to act as reservoirs of pathogens that can lead to indirect transmission. Moreover, treating a single garment with a germicidal composition fails to address the time lag between insult/inoculation with pathogens and effective reduction in numbers of pathogens.

### SUMMARY OF THE INVENTION

The present invention is directed to a germicidal surface-covering assembly that includes at least two different donnable or drapable garments. Desirably, the assembly includes at least three different donnable or drapable garments. Each garment defines at least one treated surface that is susceptible to pathogen contamination in a physical contamination event when used as intended in an environment subject to contamination (e.g., a clinical environment, a laboratory or a workplace). The treated surface is typically oriented outwardly away from a user's body and toward the environment or the source of contamination.

According to the invention, each treated surface is adapted to provide a time-dependent reduction in the number of pathogens available at that treated surface after a physical contamination event, such that at least a predetermined time after a physical contamination event at a first location on a first treated surface of a first garment, a first physical contact between the first location on the first treated surface and a second location on a second treated surface of a second garment results fewer viable pathogens on the second treated surface as compared to an untreated control. Moreover, at least a predetermined time after the first physical contact, a second physical contact between the second location on the second treated surface and a third location on a third treated surface of a third garment results in fewer viable pathogens on the third treated surface as compared to an untreated control.

According to an aspect of the invention, the germicidal surface-covering assembly includes garments may be selected from gloves, gowns, facemasks, head covers, shoe covers, surgical drapes, surgical fenestrations or coves, sheets, linens, padding, or the like.

According to the invention, the surface of the germicidal surface-covering assembly may be treated with a germicide selected from, but not limited to, one or more of: polyhexamethylene biguanide (PHMB), other biguanide compounds, chlorohexidine, alexidine, and relevant salts thereof, a quaternary ammonium compound, a quaternary siloxane, a polyquaternary amine; metal-containing species and oxides thereof, either in particle form or incorporated into a support matrix or polymer; halogens, a halogen-releasing agent or halogen-containing polymer, a bromo-compound, a chlorine dioxide, a thiazole, a thiocynate, an isothiazolin, a cyanobutane, a dithiocarbamate, a thione, a triclosan, an alkylsulfosuccinate, an alkyl-amino-alkyl glycine, a dialkyl-dimethyl-phosphonium salt, a cetrimide, hydrogen peroxide, 1-alkyl-1,5-diazapentane, cetyl pyridinium chloride, stabilized peroxide, sulfides, bis-phenols, polyphenols, chitosan, anatase TiO₂, tourmaline, hydrotropes, chaotropic agents, and synergistic combinations thereof. The germicide may be present on the germicidal garment substrate at a final concentration or add-on in a range of about 0.05-5 weight percent.

In an embodiment of the present invention, the germicidal surface-covering assembly may be formed of material having a fluid barrier property characterized, as measured by hydrostatic head testing (100 cm sample, 1 millibar/sec ramp, unsupported), of equal to or greater than about 20 millibars. For example, the germicidal surface-covering assembly may be formed of material having a fluid barrier property characterized, as measured by hydrostatic head testing (100 cm² sample, 1 millibar/sec ramp, unsupported), of equal to or greater than about 50 millibars.

In an aspect of the invention, the germicidal surface-covering assembly will reduce the contact transfer or indirect transmission from the first location to ultimately generate a reduction in viable pathogens on other surfaces such as, for example, the third treated surface. This reduction is at least a 1 log₁₀ CFU reduction of a broad spectrum of microorganisms within about 40 to about 60 seconds of initial contact, under ambient conditions as compared to an untreated control. Of course, greater reductions may occur over longer periods of time. Desirably, the germicidal surface-covering assembly will reduce the contact transfer or indirect transmission from the first location to ultimately generate a reduction in viable pathogens on other surfaces such as, for example, the third treated surface by at least a 2 log₁₀ CFU reduction within a period of about 40 to about 60 seconds after contact, as compared to an untreated control. As yet another example, the germicidal surface-covering assembly will reduce the contact transfer or indirect transmission from the first location to ultimately generate a reduction in viable pathogens on other surfaces such as, for example, the third treated surface by at least a 3 log₁₀ CFU reduction within a period of about 40 to about 60 seconds after contact, as compared to an untreated control. The microorganisms generally may include at least one of the following: Staphylococcus aureus, Enterococcus faecalis, Pseudomonas aeruginosa, Moraxella catarrhalis, Klebsiella pneumoniae, or Candida albicans. Generally speaking, the reduction in viable pathogens should take place at least 40 seconds after the physical contamination event. Desirably, the reduction in viable pathogens should take place at least 40 to about 60 seconds after the physical contamination event. Greater reductions in viable pathogens may take place over longer periods of after the physical contamination event. For example, it is contemplated that greater reductions in viable pathogens will take place over minutes, tens of minutes or even hours.

Another aspect of the present invention addresses a method of providing a germicidal surface-covering assembly. The method includes at least the following steps:
(a) providing at least two different donnable and/or drapable garments, each garment defining at least one treated surface that is susceptible to pathogen contamination in a physical contamination event when used as intended in an environment subject to contamination (e.g., a clinical environment, a laboratory, a workplace or similar location), each treated surface adapted to provide a time-dependent reduction in the number of pathogens available at that treated surface after a physical contamination event;
(b) donning at least one donnable garment on at least one individual in the environment subject to contamination, and/or draping at least one drapable garment on at least one surface in the environment subject to contamination; such that:
at least a predetermined time after a physical contamination event at a first location on a first treated surface of a first garment, physical contact between the first location on the first treated surface and a second location on a second treated surface of a second garment results fewer viable pathogens on the second treated surface as compared to an untreated control; and
at least a predetermined time after the first physical contact, a second physical contact between the second location on the second treated surface and a third location on a third treated surface of a third garment results in fewer viable pathogens on the third treated surface as compared to an untreated control.

Yet another aspect of the present invention addresses method of providing a germicidal surface-covering garment assembly which includes the following steps:
(b) providing at least two different donnable garments, each garment defining at least one treated surface that is susceptible to pathogen contamination in a physical contamination event when used as intended in an environment subject to contamination (e.g., a clinical environment, a laboratory, a workplace or similar location), each treated surface adapted to provide a time-dependent reduction in the number of pathogens available at that treated surface after a physical contamination event; and
(b) donning two or more donnable garments on at least one individual; such that:
   at least a predetermined time after a physical contamination event at a first location on a first treated surface of a first garment, physical contact between the first location on the first treated surface and a second location on a second treated surface of a second garment results fewer viable pathogens on the second treated surface as compared to an untreated control; and
   at least a predetermined time after the first physical contact, a second physical contact between the second location on the second treated surface and a third location on a third treated surface of a third garment results in fewer viable pathogens on the third treated surface as compared to an untreated control.

Additional features and advantages of the present protective and/or sanitizing articles and associated methods of manufacture will be disclosed in the following detailed description. It is understood that both the foregoing summary and the following detailed description and examples are merely representative of the invention, and are intended to provide an overview for understanding the invention as claimed.

### BRIEF DESCRIPTION OF FIGURES

FIG. 1 graphically shows an exemplary relationship between germicidal treatments of one or more garments of an exemplary germicidal surface-covering assembly.

FIG. 2 graphically shows an exemplary relationship between germicidal treatments of one or more garments of an exemplary germicidal surface-covering assembly.

### DETAILED DESCRIPTION OF THE INVENTION

In this specification and the appended claims, the singular forms “a,” “an,” and “the” include plural reference unless the context clearly dictates otherwise. Unless defined otherwise, all technical and scientific terms used herein have the same meaning as commonly understood or generally accepted by one of ordinary skill in the art to which this invention pertains.

As used herein, the terms “germicidal agent” or “germicidal agents” refer to chemicals or other substances that either kill or slow the growth of microbes. Among the germicidal agents in use today are antibacterial agents (which kill bacteria), antiviral agents (which kill viruses), antifungal agents (which kill fungi), and antiparasitic drugs (which kill parasites). A main category of germicidal agents are surface disinfectants, otherwise known as “biocides.”

The term “biocides” is a general term describing a chemical agent, such as a pesticide, usually broad spectrum, which inactivates living microorganisms. Because biocides range in germicidal activity, other terms may be more specific, including “-static,” referring to agents that inhibit growth (e.g., bacteriostatic, fungistatic, or sporistatic) and “-cidal,” referring to agents that kill the target organism (e.g., bactericidal, fungicidal, sporicidal, or virucidal). Biocides have multiple targets and modes of action, which for instance, may include physical disruption and permanent damage to the outer cell membrane of a bacterial microbe. Some example of useful biocide chemistries include biguanides (e.g., : chlorohexidine, alexidine, polyhexamethylene biguanide, and relevant salts thereof), halogen-releasing agents (e.g.,: iodine, iodophors, sodium hypochlorite, N-halamine, etc.), stabilized oxidants such as chlorine dioxide, stabilized peroxide (e.g., urea peroxide, mannitol peroxide) metal-containing species and oxides thereof (e.g., : silver, copper, selenium, etc. either in particle form or incorporated into a support matrix such as a zeolite or polymer), sulfides (e.g., sodium metabisulfite), bis-phenols (e.g., triclosan, hexachlorophene, etc), quaternary ammonium compounds (e.g., benzalkonium chloride, cetrimide, cetylpyridium chloride, quaternized cellulose and other quaternized polymers, etc.), various “naturally occurring” agents (e.g., polyphenols from green or black tea extract, citric acid, chitosan, anatase TiO₂, tourmaline, bamboo extract, neem oil, etc.), hydrotropes (e.g., strong emulsifiers) and chaotropic agents (e.g., alkyl polyglycosides) and synergistic combinations thereof. Depending on substrate chemistry (polyolefin vs. cellulosic-based materials) and the method of incorporation into the product (topical vs. grafting), many of the above chemistries could be used alone or in concert to achieve the final claimed product properties of interest.

As used herein, the term “containing” refers to the product generated according to any method of incorporating a germicidal agent into a desired item. This can include melt addition of the active agent to a polymer melt during extrusion and spinning of fibers and manufacturing of nonwoven materials used in making products; topical application methods that may or may not impart “sidedness” to the fabrics used in constructing the finished products; and other non-standard methods such as plasma treatment, electrostatic attachment, radiation surface graft coplymerizations using for example UV, gamma rays and electron-beam radiation sources, or the use of chemical initiation to produce graft copolymerized surfaces having anti-microbial activity, etc.

As used herein, the phrase “broad spectrum of microorganisms,” is defined to include at a minimum Gram positive and Gram negative bacteria, including resistant strains thereof, for example methicillan-resistant Staphylococcus aureus (MRSA), vancomycin-resistant Enterococci (VRE) and penicillin-resistant Streptococcus pneumoniae (PRSP) strains. Preferably, it is defined to include all bacteria (Gram+, Gram− and acid fast strains) and yeasts such as Candida albicans. Most preferably, it is defined to include all bacteria (Gram+, Gram−, and acid fast), yeasts, and both envelope and naked viruses such as human influenza, rhinovirus, poliovirus, adenovirus, hepatitis, HIV, herpes simplex, SARS, and avian flu.

As used herein, the phrase “results in fewer viable pathogens on a treated surface compared to an untreated control surface” and the phrase “prevents or minimizes the contact transfer” are both defined to mean that the item in question will lead to at least a 1 log₁₀ reduction in the transfer of a broad spectrum of viable microorganisms when contacting another surface as compared to an untreated control item as measured by the contact transfer protocol generally outlined in U.S. Patent Application Publication No. 2004/0151919, incorporated herein by reference with respect to the protocol, and described further in the Examples. Desirably, it leads to a reduction in viable microorganisms transfer by a factor of log₁₀ 2. More desirably, it leads to a reduction in viable microorganisms transferred by a factor of log₁₀ 2 or greater.

A “non-leaching” germicidal surface is one that passes ASTM E2149-01 testing protocol entitled “Standard Test Method for Determining the Antimicrobial Activity of Immobilized Antimicrobial Agents Under Dynamic Contact Conditions.” The lack of a zone of inhibition with the treatment agents chosen demonstrates the active species do not leach from the treated substrate.

As used herein, the term “garment” refers to articles that may be donned or worn by a person or may be laid across, shrouded or draped over a person. Garments may include gloves, head covers, shoe covers, gowns, protective apparel, drapes (including surgical drapes), sheets, linens, padding or other similar articles. A donnable garment is a garment that is adapted to be put on and worn by a wearer. Exemplary donnable garments include, but are not limited to, gowns, gloves, head covers, shoe covers, protective jackets, coveralls and other protective apparel. A drapable garment is a garment that is adapted to cover, shroud, fall around or otherwise conceal a person or article. Exemplary drapable garments include surgical drapes that are placed over a patient's body during an examination or operation.

### Description

The following description is presented to show steps generally thought to be involved in indirect transmission of pathogens and how it can be prevented by using the germicidal surface-covering assembly or system provided. This description will also demonstrate how multiple devices used together will offer a cumulative or synergistic benefit over a single device alone.

The first step in indirect transmission involves a health care worker (HCW) touching a contaminated surface with their gloved hand and then touching and thereby contaminating another article being worn (e.g., gown, mask or other item). For example, a worker with contaminated gloves may adjust a face mask or touch part of a protective garment. Alternatively, the microbes may contaminate the article worn on the health care worker by being splashed, sprayed or blown onto the article without the initial glove contact. The second step involves the HCW touching (or retouching) the contaminated part of the article to recontaminate or further contaminate their gloves (or to contaminate a new pair of gloves) and then touching a patient (or another surface) to spread the microbes. While touching or retouching of the contaminated part of the article can occur anytime, it more typically takes place within minutes (often within less than one minute) after the initial contamination. The invention involves the use of a combination of gloves, gowns, facemasks, head covers, shoe covers, drapes and/or other garments or articles treated to kill or bind pathogens that can be characterized as an assembly, system or bundle of germicidal articles that work together to minimize the indirect transmission of microbes. The assembly, system or bundle works by interrupting the second step of the indirect transmission. That is, the germicidal treatment on the articles reduces the number of microbes present on the contaminated article to reduce the chances that a new glove will get contaminated or that a germicidal-treated glove will get recontaminated to spread the microbes. As the amount of time after the microbes are first splashed, sprayed or transferred from a contaminated glove onto the article increases, so does the reduction in number of microbes. Lower levels of transfer are seen after 40 seconds. After 5 or 10 minutes, the levels of microbes are at very low levels. Although individually germicidal treated articles are known, the focus of these treatments has been to protect the wearer of the articles.

The present invention provides a germicidal surface-covering assembly or system that combines treated gloves, masks, gowns or other surface covering garments or articles to reduce indirect transmission or the contact transfer of pathogens.

As discussed above, a key component of the system will be a combination of medical devices enhanced with a germicidal agent.

### Germicidal Compositions

The germicidal compositions utilized may be one or more germicidal reagents. These reagents may be effective by themselves or may be combined to produce a synergistic effect that is non-additive of the individual components. These germicidal reagents may be further combined with processing aids and/or other ingredients that provide functional properties to the compositions. Exemplary germicidal compositions may be based on cationic polymers, such as quaternary ammonium compounds and polymeric biguanides, alcohols, and surfactants. Combinations of cationic polymers such as quaternary ammonium compounds (e.g., quaternary ammonium cellulose and quaternary ammonium siloxane), polymeric biguanides, surfactants, alcohols, and organic acids, such as acetic, citric, benzoic acids, may produce non-additive, synergistic systems with broad pathogen efficacy. The combinations with other germicidal compounds, surfactants, appear to improve germicidal efficacy of polymeric biguanides over treatments with that employ polymer biguanides alone.

Poly-hexamethylene biguanide (PHMB) hydrochloride is an exemplary cationic biguanide that is useful for providing germicidal surface-covering assemblies.

Commercially available versions of PHMB, such as under the trade names Cosmocil CQ (20 wt. % PHMB in water) or Vantocil, a heterodisperse mixture of PHMB with a molecular weight of approximately 3,000 grams/mole, are active against gram-positive and gram-negative bacteria, but may not be sporicidal.

Additional active germicidal agents may include, but are not limited to, a quaternary ammonium compound, a quaternary ammonium siloxane, a polyquaternary amine; metal-containing species and oxides thereof, either in particle form or incorporated into a support matrix or polymer; halogens, a halogen-releasing agent or halogen-containing polymer, a bromo-compound, a chlorine dioxide, a thiazole, a thiocynate, an isothiazolin, a cyanobutane, a dithiocarbamate, a thione, a triclosan, an alkylsulfosuccinate, an alkyl-amino-alkyl glycine, a dialkyl-dimethyl-phosphonium salt, a cetrimide, hydrogen peroxide, 1-alkyl-1,5-diazapentane, or cetyl pyridinium chloride.

Table 1 summarizes various biocides and processing aids that may be used in germicidal compositions that can be used to make the germicidal surface-covering assembly. It also lists their common chemical names or commercial names. Quaternary ammonium compounds, such as commercially available under the names of Aegis™ AEM 5700 (Dow Coming, Midland, Mich.) and Crodacel QM (Croda, Inc., Parsippany, N.J.), with certain surfactants such as alkyl-polyglycosides, available commercially under the name Glucopon 220 UP (Cognis Corp, Ambler, Pa.), and chitosan glycolate, available under the name Hydagen CMF and Hydagen HCMF (Cognis Corp., Cincinnati, Ohio), can significantly enhance the killing efficacy of PHMB in a synergistic fashion as will be demonstrated in the tables herein. One should note that many of the biocides described herein may be used singly or in combination in a variety of products which vary considerably in activity against microorganisms.

TABLE 1
Table of Active Reagents and Processing Aids

| | Concentration | | |
|---|---|---|---|
| Reagent | Range (wt. %) | Brand or Common Name | Vendor Name |
| Polyhexamethylene biguanide (PHMB) | 0.01-20 | Cosmocil CQ | Arch Chemicals, Inc. |
| | | | Norwalk, CT |
| Chitosan glycolate | 0.01-10 | Hydagen CMF and HCMF | Cognis Corp., |
| | | | Ambler, PA |
| Octadecylaminodimethyl Trimethoxysilylpropyl | 0.01-10 | AEGIS AEM 5700 | Dow-Corning, |
| Ammonium Chloride | | | Midland, MI |
| N-Alkyl Polyglycoside | 0.01-10 | Glucopon 220 UP | Cognis Corp., |
| | | | Ambler, PA |
| PG-Hydroxyethylcellulose Cocodimonium | 0.01-10 | Crodacel QM | Croda Inc., |
| Chloride (Quaternary Ammonium | | | Persipanny, NJ |
| CellulosicSalt) | | | |
| Xylitol | 0.01-10 | Xylitol | Sigma-Aldrich, |
| | | | Milwaukee, WI |
| 2-hydroxy-1,2,3-propanetricarboxylic acid | 0.01-10 | Citric Acid | Hach Company |
| | | | Ames, IA |
| Benzenecarboxylic acid | 0.1-2.0 | Benzoic acid | Mallinckrodt Baker, Inc |
| | | | Phillipsburg, NJ |
| 2-hydroxybenzoic acid | 0.01-10 | Salicylic acid | Mallinckrodt Baker, Inc |
| | | | Phillipsburg, NJ |
| Methane-carboxylic acid | 0.01-2.0 | Acetic acid | Sigma-Aldrich |
| | | | St. Louis, MO |
| 1,3-Propanedicarboxylic Acid | 0.01-10 | Glutaric acid | Sigma-Aldrich |
| | | | St. Louis, MO |
| Iodine | 0.05-10 | Iodine | Sigma-Aldrich |
| | | | St. Louis, MO |
| Ethyl Hydroxyethyl cellulose | 0.01-5.0 | Bermocoll EBS 481 FQ | Akzo Nobel, Inc., |
| | | (“E 481”) | Stamford, CT |
| Polyvinyl pyrrolidone | 0.01-10 | Plasdone K90 | ISP Technologies, Inc., |
| | | | Wayne, NJ |
| Poly(vinyl pyrrolidone-co-vinyl acetate) | 0.01-10 | PVP/VA S-630 | ISP Technologies, Inc., |
| | | | Wayne, NJ |
| Polyvinyl pyrrolidone-Iodine complex | 0.01-10 | PVP-Iodine | ISP Technologies, Inc., |
| | | | Wayne, NJ |
| Guanidine Hydrochloride and Sorbitol | 0.01-5.0 | Nicepole FL | NICCA USA, Inc. |
| | | | Fountain Inn, SC |
| Acrylic Co-Polymer Compound and Isopropyl | 0.01-5.0 | Nicepole FE 18U | NICCA U.S.A., Inc. |
| Alcohol | | | Fountain Inn, SC |
| 25% Copper oxide (CuO, Cu₂O) | 0.01-20.0 | Cupron* | Cupron, Inc. |
| (CAS #1317-39-1), 75% polypropylene | | | Greensboro, NC |
| (PP) resin | | | |
| Silver Sodium Hydrogen Zirconium | 0.01-20.0 | AlphaSan ® RC 2000* | Milliken, |
| Phosphate | | | Spartanburg, SC |
| Silver Zinc glass (70-100%,) barium sulfate | 0.01-20.0 | Irgaguard B 7520* | Ciba Specialty Chemicals Corp. |
| (1-30%), PP resin (10-30%) | | | Tarrytown, NY |
| *Used as internal melt additives. These additives are typically compounded in thermoplastic | | | |
| # resins (e.g., polypropylene (PP)) to produce a concentrate which is then dry blended with the | | | |
| # virgin resin and co-extruded to produce fibers and webs containing such additives. The additive | | | |
| # is generally distributed throughout the bulk of the fiber and enough of the additive is present | | | |
| # on the surface of the fiber to provide anti-microbial activity. Concentration of the additive | | | |
| # present on the surface of the fiber depends on several factors including additive concentration | | | |
| # in the melt relative to the main body of resin or type of resin, processing | | | |
| # conditions and thermal history, crystallinity of the resin, and | | | |
| # relative thermodynamic compatibility of the resin and the additive. It is understood that | | | |
| # the additive must be compatible with thermoplastic resin in the melt for proccesability, and | | | |
| # yet it is desirable that the additive be less compatible with the resin at ambient conditions | | | |
| # so that the additive migrate to a certain extent to the surface of the thermoplastic | | | |
| # fiber. Processing aids such as amorphous compounds can be added to the main resin to ease migration | | | |
| # of the additive to the fiber surface. It is also understood that other active ingredients | | | |
| # such as PHMB can be compounded and co-extruded in various other thermoplastic resins. | | | |

Table 2 summarizes a number of illustrative compositional examples that may be used to make the germicidal surface-covering assembly and provides various percent combinations of reagents listed in Table 1. Each reagent is presented in terms of weight percent (wt %) of the active agents in the total formulation. Other components such as processing aids (e.g., hexanol, octanol, alkyl-polyglycoside, or other surfactants) to enhance wetting and/or treatment coating uniformity can be incorporated into the formulation in a range from about 0.1 to about 1 wt %, with respect to the total amount of ingredients in the composition. In certain embodiments, the processing aids are present in about 0.2- 0.75 wt % concentration. The respective formulations may be mixed in an aqueous solution. The formulation can be diluted to any desired or required concentration level, depending on the treatment process to achieve the desired or predetermined add on amount on a substrate for germicidal efficacy.

The individual components are listed using the common or commercial brand name only as a shorthand form to identify the individual chemical reagents, and should not be construed to limit the invention to any particular commercial embodiment or formulation. The compositional examples of Table 2, all can be used as topical coatings over a predetermined organic or inorganic substrate, and each is generally thought to be effective in producing about at least a 1 log₁₀ reduction in the colony forming units (CFU/mL)(CFU/g) within about 60 seconds. Desirably, the compositions serve to quickly kill microbes within about 10 minutes, and in some cases within 5 minutes.

While PHMB is a constituent of all of the compositions in Table 2, various other combinations of ingredients that do not include PHMB may be utilized to carry out the germicidal surface-covering assembly of the present invention.

In certain embodiments the germicidal composition includes combinations of biocide active agents that work against both bacteria and viruses. For instance, a composition may include: PHMB, quaternary ammonium cellulose, xylitol, citric acid, benzoic acid, surfactant, complexing agent (e.g., PVP), and/or antistatic agent (e.g., Nicepole FL). A desirable antistatic agent is one that does not reduce surface tension of water by more than 20 dynes/cm. The present composition desirably is moderately hydrophilic; hence, a droplet of a formulation applied to a surface can produce a contact angle of less than about 90° with respect to, for example, a polypropylene substrate surface. The compositions have a pH in a range of about 2 to about 5 or 6. Preferred pH ranges are about 2.5-4, or 2.5-3.5, depending on the desired, particular environmental conditions for use. The compositions may also contain an acrylic co-polymer compound and isopropyl alcohol, which serves as an antistatic agent useful for treating nonwoven fabrics such as those commonly found in medical fabrics.

A germicidal solution may contain a primary active agent, for example, 0.1-99.9 wt % polyhexamethylene biguanide (PHMB) by weight of active agents, and a secondary active agent selected from at least one of the following: alkyl polyglycosides, quaternized cellulose derivatives, quaternized siloxanes, surfactants, and organic acids. The final concentration for each of the active reagent and processing aids on a treated substrate can range from about 0.01-20 wt %. The exact concentrations may depend on the specific kind of microorganism that one is targeting against and/or the nature of the coated substrate material. As an illustration, the general concentration ranges for each component individually in the examples are summarized in Table 2.

TABLE 2
Concentration of Composition Components on a Treated substrate.

| | Target |
|---|---|
| | Concentration |
| Reagent | (wt %) |
| Polyhexamethylene biguanide (PHMB) | 0.01-5 |
| Chitosan glycolate | 0.01-4 |
| Octadecylaminodimethyl Trimethoxysilylpropyl | 0.01-4 |
| Ammonium Chloride | |
| Alkyl Polyglycoside | 0.01-1 |
| PG-Hydroxyethylcellulose Cocodimonium Chloride | 0.01-1.5 |
| (Quaternary Ammonium Cellulosic Salt) | |
| Xylitol | 0.01-1.5 |
| 2-hydroxy-1,2,3-propanetricarboxylic acid | 3-8.5 |
| Benzenecarboxylic acid | 0.3-0.7 |
| 2-hydroxybenzoic acid | 0.5-3.5 |
| Ethanoic acid | 0.5-3.5 |
| 1,3-Propanedicarboxylic Acid | 0.5-3.5 |
| Iodine | 1-2 |
| Ethyl Hydroxyethyl cellulose | 0.05-0.5 |
| Polyvinyl pyrrolidone | 0.05-1.5 |
| Poly(vinyl pyrrolidone-co-vinyl acetate) | 0.05-1.5 |
| Polyvinyl pyrrolidone-Iodine complex | 0.05-1.5 |
| Guanidine Hydrochloride and Sorbitol | 0.03-1.5 |
| Acrylic Co-Polymer Compound and Isopropyl Alcohol | 0.03-1.5 |
| 25% Copper oxide (CAS #1317-39-1) | 0.1-5.0 |
| 75% PP resin | |
| Silver Sodium Hydrogen Zirconium Phosphate | 0.1-5.0 |
| Silver Zinc glass (70-100%,) barium sulfate (1-30%), PP | 0.1-5.0 |
| resin (10-30%) | |

The germicidal composition should be odorless to humans; that is, the composition is undetectable at least to the human olfactory system. This characteristic is important if the germicidal composition is to be used on face masks and other substrates that come into close proximity to the human nose.

### Substrates

A variety of different kinds of substrates can be treated or coated with the germicidal compositions to generate the base materials, finished garments or other articles forming the germicidal surface-covering assembly of the present invention. According to certain embodiments, the substrate materials may include, for example, elastomeric membranes, films or foams, such as natural rubber or synthetic polymer latex, soft rubber or plastic surfaces, such as found with typical donnable and/or drapable garments or articles. It is contemplated that the assembly may be also used in combination with germicidal medical devices and/or surgical equipment and instruments, or hospital physical plant. Alternatively, other embodiments may have substrate materials that are selected from either woven or nonwoven fabrics that can be used to form donnable and/or drapable garments or articles. Woven fabrics may be made from natural fibers (e.g., cellulose, cotton, flax linen, wool, silk) or a blend of natural and synthetic fibers (e.g., thermoplastics, polyolefin, polyester, nylon, aramide, polyacrylic materials). A wide variety of elastic or non-elastic thermoplastic polymers may be used to construct nonwoven substrate materials. For example, without limitation, polyamides, polyesters, polypropylene, polyethylene, copolymers of ethylene and propylene, polylactic acid and polyglycolic acid polymers and copolymers thereof, polybutylene, styrenic co-block polymers, metallocene-catalyzed polyolefins, preferably with a density of less than 0.9 gram/cm³, and other kinds of polyolefins, for the production of various types of elastic or non-elastic fibers, filaments, films or sheets, or combinations and laminates thereof.

The beneficial attributes of the present invention may be illustrated with donnable and/or drapable garments or articles fashioned from nonwoven materials treated with the germicidal compositions described above. Treated nonwoven fabrics can be made into a variety of products, which may include, for example, various protective garments, gowns or aprons, and industrial wear, as well as sheet materials that can be used in the manufacture of bedding fabrics, fenestration covers, wraps, or pads. Other uses may be for various articles, such as face masks, hand gloves, or foot covers, which may have medical uses, industrial uses or both. In addition, the germicidal surface-covering assembly may be used in conjunction with germicidal wipers or the like.

Generally speaking, the treated surface of the germicidal surface-covering assembly should be outward or exterior facing and away from the skin-contacting surface such as a lining of a garment or article. The purpose of this orientation is to address the indirect transmission or the contact transfer of pathogens.

Generally speaking, nonwoven materials treated with the germicidal compositions should largely maintain their liquid barrier properties when segregated to the surface of the materials. It is believed that by means of controlling the topical placement of the germicidal composition, in which germicidal agents are confined to the outermost or top spunbond layer of a SMS substrate, for instance, one can prevent the creation of a liquid conduit into the under layers of the substrate material, thereby achieving the beneficial combination of barrier and germicidal properties. Desirably, the substrate attains a measure of barrier protection performance of ≧20 millibars hydrostatic head pressure (100 cm² sample, 1 millibar/sec ramp, unsupported).

In addition, placing the germicidal chemistry on the surface of the substrate will make the biocides more readily available to interact with pathogens, thus improving overall efficacy.

In certain embodiments of the present invention, the coated nonwoven material substrate may impart antistatic properties when an antistatic agent, such as an acrylic copolymer and isopropyl alcohol or guanidine hydrochloride and sorbitol, is added to the germicidal composition.

Embodiments of the germicidal surface-covering assembly include donnable and/or drapable garments such as gloves, face masks, surgical or medical gowns, drapes, shoe covers, or fenestration covers or other protective articles. With respect to a facemask containing one or more germicidal agents, the germicidal coating can be selectively placed on the exterior nonwoven facing of the mask rather than throughout the entire product. The germicidal agents are desirably non-leaching from the surface of the mask in the presence of fluids, and/or not recoverable on particles that may be shed by the mask in use and potentially inhaled by the user as measured using conventional blow-through test protocols.

With respect to germicidal cover gowns, the germicidal agents covering the gown surface should be stably associated with the substrate and desirably non-leaching from the surface of the gown in the presence of fluids. The gown can possess a fluid barrier characteristic, as measured by hydrostatic head testing, of equal to or greater than about 20 millibars (100 cm² sample, 1 millibar/sec ramp, unsupported). Preferably, the fluid barrier is measured to be equal or greater than about 50 millibars (100 cm² sample, 1 millibar/sec ramp, unsupported). More preferably, the gown fabric is also resistant to blood and viral penetration, as defined by test standards ASTM F1670 and ASTM F1671. The fluid barrier can be equal to or greater than about 100 millibars (100 cm² sample, 1 millibar/sec ramp, unsupported).

Preferably, the germicidal-treated gowns can dissipate 50% of a 5000V electrostatic charge in less than 0.5 seconds as measured by static decay testing using the Association of the Nonwovens Fabrics Industries (INDA) Standard Test Method 40.2 (95). In addition, the gown material desirably has a Class I flammability rating as measured by flame propagation protocol (CPSC 1610 and NFPA 702). Both the static decay and flame propagation requirements are critical in a hospital setting to minimize the potential likelihood of a fire due to accidental static discharge. It is important to note that not all choices of substrate and germicidal composition will lead to this advantageous set of properties and codes that pass both of these criteria in addition to possessing germicidal properties are preferred embodiments.

### Process Methods

The germicidal compositions can be applied topically to the external surfaces of nonwoven web filaments after they are formed. Desirably, a uniform coating is applied over the substrate surfaces. A uniform coating refers to a layer of germicidal agents that does not aggregate only at selected sites on a substrate surface, but has a relatively homogeneous or even distribution over the treated substrate surface. Desirably, the processing aid should evaporate or flash off once the germicidal composition dries on the substrate surface. Suitable processing aids may include alcohols, such as hexanol or octanol. Note that the terms “surface treatment,” “surface modification,” and “topical treatment” refer to an application of the present germicidal formulations to a substrate and are used interchangeably, unless otherwise indicated.

Nonwoven fabrics that are treated with a germicidal coating can be fabricated according to a number of processes. In an illustrative example, a method for preparing an anti-microbial treated substrate involves providing a hydrophobic polymer substrate and exposing at least a portion of the substrate to a mixture that includes at least one anti-microbial active agent (e.g. PHMB) and may optionally include one or more co-active agent (e.g. AEGIS AM 5700) and/or processing aid (e.g. alky-polyglycoside, or other surfactants). A suggested combination includes contacting the substrate with a mixture that includes an anti-microbial agent, a wetting agent, a surfactant, and a rheology control agent. These components of the treatment composition may be combined in water mixture and applied as an aqueous treatment. The treatment composition may further include other components, such as anti-stats, skin care ingredients, anti-oxidants, vitamins, botanical extracts, scents, odor control agents, low surface tension fluid repellent chemistries, and colors. The final amount of active reagents on the treated substrate may be diluted to a desired or predetermined concentration.

According to an embodiment, the germicidal composition can be applied to the material substrate via conventional saturation processes such as a so-called “dip and squeeze” or “padding” technique. The “dip and squeeze” or “padding” process can coat both sides of and/or through the bulk of the substrate with the germicidal composition. When dipped in a bath, the germicidal solution be a unitary medium containing all components, or in subsequent multiple step processing, other desired components may be later added to the base germicidal layer. For instance, a formulation of a unitary germicidal solution may include leveling and/or antistatic agents. On substrates containing polypropylene, an antistatic agent can help dissipate static charge build-up from mechanical friction. An antistatic agent can be added to the germicidal solution, and the mixture can be introduced simultaneously to the material substrate in one application step. Alternatively, the antistatic solution can be applied using a spray after the germicidal solution in a second step.

In certain product forms, where one wishes to treat only a single side and not the inner layers or opposing side of the sheet substrate, in which the substrate material is layered to another sheet ply (e.g., filter or barrier media) that is without the germicidal treatment, other processes are preferred such as at rotary screen, reverse roll, Meyer-rod (or wire wound rod), Gravure, slot die, gap-coating, or other similar techniques, familiar to persons in the nonwoven textile industry. (See, for example, detailed descriptions of these and other techniques are available from Faustel Inc., Germantown, Wis. (www.faustel.com).) Also one may consider printing techniques such as flexographic or digital techniques. Alternatively one may use a combination of more than one coating to achieve a controlled placement of the treatment composition. Such combination may include, but not limited to, a reverse Gravure process followed by a Meyer rod process. Alternatively, the germicidal composition may be applied through an aerosol spray on the substrate surface. The spray apparatus can be employed to apply the germicidal solution and/or antistatic agent only on one side of the substrate sheet or on both sides separately if desired. An antistatic agent can be applied to the substrate in a secondary step, for example, using a spray system or any other conventional application process. On sheet materials, the treated nonwoven substrates can achieve at least a hydrostatic head greater than 20 millibars (100 cm² sample, 1 millibar/sec ramp, unsupported). Germicidal coatings are applied in as at least a single layer over SMS fabrics. Alternatively, one can use a melt extrusion process to incorporate a germicidal agent into the material followed by topical application of a second anti-microbial agent or co-active from an aqueous solution. Furthermore, other ingredients can also be added during the melt extrusion to enhance for example: a) wettability of the material if desired, b) electrical conductivity or anti-static properties, c) skin emollient, d) anti-oxidants, etc.

Various other methods may be employed for contacting a substrate with the treatment composition or compositions in accordance with the invention. For example, a substrate may be printed on by means of print rolls or other coating steps, or spray techniques may be employed. Preferably, the treatment composition or compositions are applied as an overlayer onto the substrate by a Meyer rod, reverse Gravure or flexographic techniques, for example, in such a way that the treatment composition forms a uniform and homogeneous layer on top of the substrate with minimum penetration of the treating composition into the bulk of the substrate. The overlayer coating, in general, results in more uniform distribution of the anti-microbial treatment on the substrate and permits the anti-microbial agent(s) to be more readily available on the surface of the substrate. The overlayer coating technique also results in maintaining better barrier properties of the substrate.

A nonwoven web or laminate can treated with compositions and methods of the present invention to impart broad spectrum anti-microbial and antistatic properties at desired or predetermined locations on the substrate, while maintaining desired barrier properties. Furthermore, the components of the treatment composition can be applied in separate steps or in one combined step. It should also be understood that the method and anti-microbial surface treatment of nonwoven materials with topical application of ingredients of this invention may incorporate not only multiple ingredients for improved anti-microbial performance but may also be used to incorporate anti-static agents which may afford dissipation of static charge build up.

With respect to gloves, gloves made from either woven or nonwoven textiles, leather, or elastomeric materials (e.g., natural rubber latex or synthetic polymers) can be either sprayed with a heated solution or immersed in a heated bath containing an antifoaming agent, and versions of the above-described germicidal compositions. The solution is heated by the spray atomizer or in a heated canister before entering the atomizer while tumbling in a forced air-dryer. This exemplary method allows only the outside of the glove to be treated more efficiently with less solution and still provide the germicidal efficacy desired, better adhesion of the germicidal to mitigate any leaching of the agent off the surface, and also eliminates the potential for skin irritation for the wearer due to constant contact between the biocide and the user's skin. Of course, other conventional techniques for applying the germicidal compositions to gloves may be utilized.

### Germicidal Test Method

A. Sample Preparation

Test organisms are grown in 25 mL appropriate broth medium for about 24±2 hours at 37±2° C. in a orbital shaker. The bacterial culture is then transferred by placing about 100 μL aliquot in 25mL of broth and grown again for about 24±2 hours at 37±2° C. The organisms are then centrifuged and washed three times with phosphate buffered saline (PBS). The organisms are then suspended in PBS to obtain an inoculum of approximately 1×10⁸ CFU/mL.

The test articles and control swatches are exposed to an ultraviolet light source for about 5-10 minutes per side before testing to assure that the swatches are sanitized prior to inoculation with the bacteria. The test materials are brought into contact with a known population of test bacteria from the inoculum for a specified period of time. A sample is then plated at the end of the exposure time to enumerate the surviving bacteria. The log₁₀ reduction from the control material and the original population is calculated using the following formula:
Log₁₀ Control*−Log₁₀ CFU/swatch Test Article=Log₁₀ Reduction

*CFU/swatch from control swatches or theoretical CFU/swatch.

After exposing the bacteria to the surface of a treated product for a designated amount of time (˜40 seconds), the substrate is placed in a flask and a buffer solution is added to elute the microorganisms off the substrate prior to plating them to see how many are left alive. This buffer solution contains a chemical to de-activate or “neutralize” the germicidal agent to (a) stop the active agent from killing the organisms after the designated time period and (b) to prevent artifacts that may arise from exposing the microorganisms to the germicidal in solution rather than solely on the substrate. The neutralizer is pre-screened to make sure that they do not affect the microorganisms. The neutralizer employed may be selected from a list that is commonly used in the field. These include non-ionic detergents, Bisulphate, lecithin, Leethen broth, thiosulfate, thioglycollate, and pH buffers. Method similar to those described in American Society for Testing and Materials, Standard Practices for Evaluating Inactivators of Germicidal Agents Used in Disinfectant, Sanitizer, Antiseptic, or Preserved Products, Amer. Soc. Testing Mat. E 1054-91 (1991) can be used.

B. Contact Transfer Protocol

The following generalized discussion relates to a method for determining viable microbe transmission levels or contact transfer of microbes from one contaminated article to at least one other article. Generally speaking, the method includes applying an inoculum including a microbe to a first surface, contacting a transfer substrate to the first surface, extracting the transferred inoculum from the transfer substrate, permitting the extracted inoculum to incubate, and quantifying the microbe level to determine a percent recovery. As used herein, “inoculum” refers to any material containing at least one microbe that may act as a source of infection in a host.

The method may be used to measure viable contact transfer of various microbes, including, for example, Aspergillus niger (American Type Culture Collection (ATCC®) No. 16404), Candida albicans (ATCC® No. 10231), Hepatits A HM175/18f (ATCC® No. VR-1402), Herpes simplex virus 1 GHSV-UL46D (ATCC® No. VR-1545), Acinetobacter baumannii (ATCC® No.15149), Clostridium difficle (ATCC® No.43594), Enterobacter cloacae (ATCC® No.29249), Enterococcus faecalis (ATCC® No.51299), Enterococcus faecium (ATCC® NO. 700221), Enterococcus hirae (ATCC® No. 10541), Escherichia coli (ATCC® No. 13706), Escherichia coli (ATCC® No. 31705), Mycobacterium smegmatis (ATCC® No. 10143), Mycobacterium tuberculosis (ATCC® 27294), Pseudomonas aeruginosa (ATCC® No. 9027), Pseudomonas aeruginosa (ATCC® No. 27853), Staphylococcus aureus (ATCC® No. 6538), Staphylococcus aureus (ATCC® No. 33592), Staphylococcus epidermidis (ATCC® No. 12228), and Staphylococcus epidermidis (ATCC® No. 51625).

After the desired microbe is selected, an inoculum is prepared by diluting a stock culture of the microbe. The culture may be diluted to any desired level using a sterile buffered liquid, and in some instances, may be diluted to an inoculum level of from about 1×10⁶ colony forming units (CFU)/ml to about 3×10⁶ CFU/ml. However, for the present testing, the inoculum level was 5×10⁸ CFU/ml.

Prior to performing the evaluation, a sterile buffer solution may be prepared for later use. The buffer solution may be replaced about every two months. In some instances, the buffer solution may be sterile phosphate buffered water.

The desired inoculum is then placed aseptically onto a first surface. Any quantity of the desired inoculum may be used. However, for the contact transfer testing of the germicidal surface-covering assembly, a quantity of about 0.5 ml is applied to the first surface. Furthermore, the inoculum may be applied to the first surface over any desired area. In some instances, the inoculum may be applied over an area of about 7 inches (178 mm) by 7 inches (178 mm). However, in the present testing, the inoculum is applied to substantially all of a 4 inch (101 mm) by 4 inch (101 mm) square piece of material that constitutes the first surface.

The inoculum is then permitted to remain on the first surface for a relatively short amount of time. For example, about 20 seconds before the article to be evaluated, i.e., the transfer substrate is brought into contact with the first surface.

The transfer substrate may be any drapable or donnable article, and in some instances, is a surgical or examination glove, surgical or cover gown, or facemask. The transfer substrate, for example, the glove, should be handled aseptically. Where the transfer substrate is a glove, a glove may be placed on the left and right hands of the experimenter. One glove may then be brought into contact with the inoculated first surface, ensuring that the contact is firm and direct to minimize error. The test glove may then be immediately removed using the other hand and placed into a flask containing a desired amount of sterile buffered water (prepared above) to extract the transferred microbes. In some instances, the glove may be placed into a flask containing about 100 ml of sterile buffered water and tested within a specified amount of time. Alternatively, the glove may be placed into a flask containing a suitable amount of Letheen Broth (available from Alpha Biosciences, Inc. of Baltimore, Md.) to neutralize the antimicrobial treatment for later evaluation. The flask containing the glove may then be placed on a reciprocating shaker and agitated at a rate of from about 190 cycles/min. to about 200 cycles/min. The flask may be shaken for any desired time, and in some instances is shaken for about 2 minutes.

The glove may then be removed from the flask, and the solution diluted as desired. A desired amount of the solution may then be placed on at least one agar sample plate. In some instances, about 0.1 ml of the solution may be placed on each sample plate.

The solution on the sample plates may then be incubated for a desired amount of time to permit the microbes to propagate. In some instances, the solution may incubate for at least about 48 hours. The incubation may take place at any optimal temperature to permit microbe growth, and in some instances may take place at from about 33° C. to about 37° C. In some instances, the incubation may take place at about 35° C.

After incubation is complete, the microbes present are counted and the results are reported as CFU/ml. The percent recovery may then be calculated by dividing the extracted microbes in CFU/ml by the number present in the inoculum in (CFU/ml), and multiplying the value by 100.

The various aspects of the present invention may be better understood with reference to the following example.

### EXAMPLE

This example was structured to assess whether germicidal treated products in combination (gloves, mask and gown) are more effective at reducing the transfer of organisms than any one product alone following purposeful contamination during simulated use. To carry this out, A piece of conventional polyolefin nonwoven face mask material was inoculated with Staphylococcus aureus ATCC 27660 suspended in 5.0% w/v bovine serum albumin (BSA) solution. A test subject handled the inoculated material with a conventional nitrile examination glove for 20 seconds, followed by handling a piece of polyolefin nonwoven gown material for 20 seconds. After contact, the test articles were placed in a neutralizer and tested for the amount of viable bacteria extracted from each material.

| Test Articles: | F_{U} = Untreated face mask material | | | | | |
|---|---|---|---|---|---|---|
| | F_{T} = Antimicrobial face mask material | | | | | |
| | G_{U} = Untreated glove | | | | | |
| | G_{T} = Antimicrobial glove | | | | | |
| | W_{U} = Untreated gown material | | | | | |
| | W_{T} = Antimicrobial gown material | | | | | |
| Scenario(s): | F →G →W | | | | | |
| Series: | 1) F_{U} → G_{U} → W_{U} | | | | | |
| | 2) F_{T} → G_{T} → W_{T} | | | | | |
| | 3) F_{T} → G_{U} → W_{U} | | | | | |
| | 4) F_{U} → G_{T} → W_{U} | | | | | |
| | 5) F_{U} → G_{U} → W_{T} | | | | | |
| Repetitions per | 14 | | | | | |
| Series: | | | | | | |
| Series Order: | | Day 1 | Day 2 | Day 3 | Day 4 | Day 5 |
| | Series | 1 | 1 | 1 | 1 | 1 |
| | Number | 1 | 1 | 1 | 1 | 1 |
| | | 1 | 2 | 1 | 1 | 1 |
| | | 2 | 2 | 2 | 2 | 2 |
| | | 2 | 2 | 2 | 2 | 2 |
| | | 2 | 3 | 3 | 2 | 2 |
| | | 3 | 3 | 3 | 3 | 3 |
| | | 3 | 3 | 3 | 3 | 3 |
| | | 3 | 4 | 4 | 4 | 3 |
| | | 4 | 4 | 4 | 4 | 4 |
| | | 4 | 4 | 4 | 4 | 4 |
| | | 4 | 5 | 5 | 5 | 5 |
| | | 5 | 5 | 5 | 5 | 5 |
| | | 5 | 5 | 5 | 5 | 5 |

| | |
|---|---|
| Amount of | 0.05 |
| Bacteria | |
| Applied (ml): | |
| Method of | Apply to the center of the test article. |
| Application: | |
| Contact | Apply inoculum to appropriate face mask material |
| instructions: | according to the series order chart. Wait for 20 sec. |
| | Grasp the test article with the appropriate gloved |
| | hand (according to the series order chart) and hold |
| | for 20 sec. Release face mask material and place it |
| | immediately into Letheen broth. Grasp the gown |
| | material and hold for 20 sec. Release the gown |
| | material and place the glove immediately into Letheen |
| | broth. Allow the gown material to sit for 20 sec then |
| | place it into Letheen broth. |

1. Randomization

Each study will follow a randomization schedule of alternating right and left hands for the handling of the test materials.

2. Procedure

Day 1 up to Day 5

2.1. The subject will don a gown, mask, goggles and a pair of snuggly fitting untreated exam gloves. The subject will apply a second glove that corresponds to the series order provided by the sponsor over an untreated exam glove.

2.2. A study assistant will inoculate a 4″×4″ square piece of test material with 0.5 ml of bacterial culture containing approximately 5×10⁸ CFU/ml of Staphylococcus aureus ATCC 27660. The inoculum will be mixed with 5.0% (weight/volume) bovine serum albumin. The material will be draped over an alcohol washed (sterile) racquet ball in a rack to prevent rolling. A fresh inoculum suspension will be prepared each day.

2.3. After 20 seconds have elapsed from the initial inoculation, the subject will grasp the test material and racquet ball for 20 seconds with the hand containing the test glove. The racquet ball is used to provide a controlled surface area for contact with the glove.

2.4. After 20 seconds have elapsed, the subject will return the test material and racquet ball to the rack. The inoculated test material (or alternatively the implement) will be immediately placed into a prelabeled specimen cup containing 75 mL Letheen broth.

2.5. The study conductor will drape a piece of sterile test material over a racquet ball in a rack. After the release of the inoculated material, the subject will immediately grasp the second test article with the same gloved hand. The subject will continue to hold the racquet ball and material for 20 seconds.

2.6. After 20 seconds have elapsed, the subject will return the ball and material (or implement) to the rack. The glove will be removed immediately and placed into a prelabeled specimen cup containing 75 mL Letheen broth.

2.7. The second test article (or implement) will be allowed to sit for 20 seconds, then placed in a prelabeled specimen cup containing 75 mL Letheen broth.

2.8. Steps 2.5 through 2.7 may be repeated with a third test material or implement.

2.9. Repeat steps 2.2 to 2.8 for according to the series order.

The following tables present illustrative examples of the beneficial effect of the present invention in comparison to untreated controls.

TABLE 3A
All untreated
Series 1

| replicate | Mask | Glove | Gown |
|---|---|---|---|
| 1 | 5.2 | 5.7 | 5.7 |
| 2 | 4.8 | 6.1 | 4.4 |
| 3 | 4.9 | 5.9 | 5.4 |
| Average | 5.0 | 5.9 | 5.2 |
| Stdev | 0.2 | 0.2 | 0.7 |

TABLE 3B
All treated
Series 2

| replicate | Mask | Glove | Gown |
|---|---|---|---|
| 1 | 3.7 | 2.7 | 1.9 |
| 2 | 3.8 | 4.3 | 4.0 |
| 3 | 3.5 | 3 | 1.9 |
| Average | 3.7 | 3.3 | 2.6 |
| Stdev | 0.2 | 0.9 | 1.2 |

TABLE 3C
Mask only Treated
Series 3

| replicate | Mask | Glove | Gown |
|---|---|---|---|
| 1 | 4.1 | 5.3 | 3.6 |
| 2 | 3.6 | 5.1 | 3.7 |
| 3 | 3.4 | 5.5 | 4.3 |
| Average | 3.7 | 5.3 | 3.9 |
| Stdev | 0.4 | 0.2 | 0.4 |

TABLE 3D
Glove only Treated
Series 4

| replicate | Mask | Glove | Gown |
|---|---|---|---|
| 1 | 5.6 | 3.1 | 3.8 |
| 2 | 5.0 | 3.7 | 4.0 |
| 3 | | n/a | |
| Average | 5.3 | 3.4 | 3.9 |
| Stdev | 0.5 | 0.5 | 0.1 |

TABLE 3E
Gown only Treated
Series 5

| replicate | Mask | Glove | Gown |
|---|---|---|---|
| 1 | 5.6 | 5.7 | 3.3 |
| 2 | 5.7 | 5.5 | 4.0 |
| 3 | 5.1 | 6.1 | 3.7 |
| Average | 5.5 | 5.8 | 3.7 |
| Stdev | 0.3 | 0.3 | 0.3 |

TABLE 4A
All untreated
Series 1

| replicate | Mask | Glove | Gown |
|---|---|---|---|
| 1 | 1.7E+05 | 4.5E+05 | 5.3E+05 |
| 2 | 5.7E+04 | 1.2E+06 | 2.5E+04 |
| 3 | 8.6E+04 | 8.4E+05 | 2.4E+05 |
| Average | 1.0E+05 | 8.1E+05 | 2.6E+05 |
| Stdev | 6.0E+04 | 3.5E+05 | 2.5E+05 |
| SUM of all articles | 1.2E+06 | 6.1 | |
| in the series | | | |

TABLE 4B
All treated
Series 2

| replicate | Mask | Glove | Gown |
|---|---|---|---|
| 1 | 4.8E+03 | 5.0E+02 | 7.6E+01 |
| 2 | 7.0E+03 | 2.2E+04 | 9.8E+03 |
| 3 | 3.5E+03 | 1.0E+03 | 7.6E+01 |
| Average | 5.1E+03 | 7.8E+03 | 3.3E+03 |
| Stdev | 1.8E+03 | 1.2E+04 | 5.6E+03 |
| SUM of all articles | 1.6E+04 | 4.2 | |
| in the series | | | |

TABLE 4C
Mask only Treated
Series 3

| replicate | Mask | Glove | Gown |
|---|---|---|---|
| 1 | 1.1E+04 | 1.8E+05 | 4.3E+03 |
| 2 | 4.3E+03 | 1.3E+05 | 5.0E+03 |
| 3 | 2.3E+03 | 3.4E+05 | 2.2E+04 |
| Average | 5.9E+03 | 2.1E+05 | 1.0E+04 |
| Stdev | 4.7E+03 | 1.1E+05 | 9.9E+03 |
| SUM of all articles | 2.3E+05 | 5.4 | |
| in the series | | | |

TABLE 4D
Glove only Treated
Series 4

| replicate | Mask | Glove | Gown |
|---|---|---|---|
| 1 | 4.0E+05 | 1.3E+03 | 6.5E+03 |
| 2 | 9.2E+04 | 5.5E+03 | 9.3E+03 |
| 3 | | n/a | |
| Average | 2.5E+05 | 3.4E+03 | 7.9E+03 |
| Stdev | 2.2E+05 | 3.0E+03 | 1.9E+03 |
| SUM of all articles | 2.6E+05 | 5.4 | |
| in the series | | | |

TABLE 4E
Gown only Treated
Series 5

| replicate | Mask | Glove | Gown |
|---|---|---|---|
| 1 | 3.6E+05 | 5.5E+05 | 2.0E+03 |
| 2 | 4.7E+05 | 3.2E+05 | 9.3E+03 |
| 3 | 1.4E+05 | 1.2E+06 | 5.0E+03 |
| Average | 3.2E+05 | 6.7E+05 | 5.4E+03 |
| Stdev | 1.7E+05 | 4.3E+05 | 3.6E+03 |

SUM of all articles in the series 1.0E+06 6.0

### Discussion

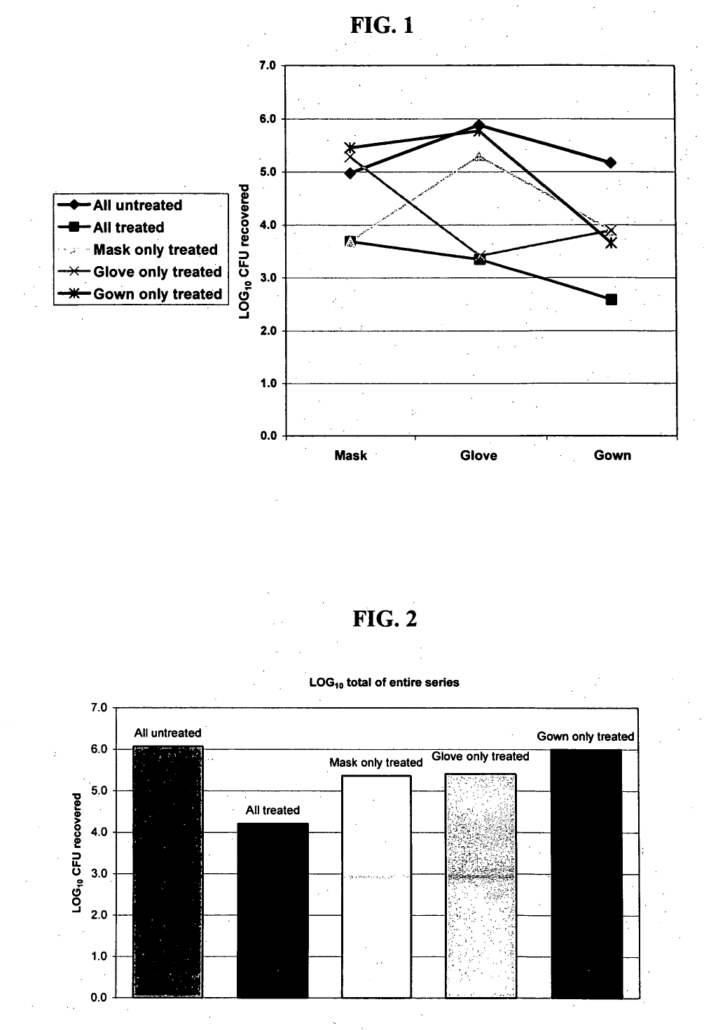

The data in Tables 3A to 3E are expressed base-10 logarithm (i.e., log₁₀). Tables 3A to 3E represent the colony forming units (CFU) recovered for each type of garment after a series of contact transfers between various combinations of garments that were or were not treated. These results are also shown graphically in FIG. 1. As shown in FIG. 1, when only a single garment of an exemplary three garment assembly has a germicidal treatment, the reduction in viable pathogens is relatively low. However, when all the garments handled in the sequence of testing an exemplary assembly have a germicidal treatment, the reduction in viable pathogens is substantial, especially as compared to the untreated control and to the examples with only one treated garment. Moreover, when all the garments handled in the sequence of testing an exemplary assembly have a germicidal treatment, the amount of pathogen (log₁₀ CFU recovered) decreases across the sequence of garments in the testing as compared to the untreated control and to the examples with only one treated garment. It is important to note that the reductions are achieved at a combined inoculation and contact times of about 40 seconds.

The data in Tables 4A to 4E are expressed in scientific notation. Tables 4A to 4E represent the colony forming units (CFU) recovered for each type of garment after a series of contact transfers between various combinations of garments that were or were not treated. Tables 4A to 4E also show the sum of colony forming units (CFU) recovered for all garments in the specific series of garments tested. These results are also shown graphically in FIG. 2.

When only a single garment of an exemplary three garment assembly has a germicidal treatment, the reduction in viable pathogens is relatively low. When only the last garment handled in the sequence of testing an exemplary assembly has a germicidal treatment (i.e., Table 4E—the gown only treated), the reduction in viable pathogens represented as the sum of colony forming units (CFU) recovered for all garments in the specific series of garments tests is nominal as compared to an untreated control. It is important to note that the reductions are achieved at a combined inoculation and contact times of about 40 seconds.

While the inventors should not be held to a particular theory of operation, it is thought that multiple germicidal treated garments (i.e., at least two treated garments of the assembly) are important for reducing the amount of viable pathogens available for indirect transfer of pathogens on the garments. Treating a single garment with a germicidal composition fails to address the time lag between insult/inoculation with pathogens and effective reduction in numbers of pathogens. By providing an assembly of germicidal garments, the grouping of these garments provides an array of germicidal surfaces that address the lag time between insult/inoculation with pathogens and effective reduction in numbers of pathogens. Even relatively small reductions of viable pathogens on a first garment cascade into increasingly lower levels of viable pathogens available for transfer in sequential contact with other germicidal treated garments. Reducing the levels of pathogens in this manner is important for reducing the indirect transmission of pathogens to others and for improving the control of infection by such pathogens.

The present invention has been described in general and in detail by way of examples. The words used are words of description rather than of limitation. Persons of ordinary skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein and the appended claims should not be limited to the description of the preferred versions herein.

We Claim:

1. A germicidal surface-covering assembly comprising:
at least two different donnable or drapable garments, each garment defining at least one treated surface susceptible to pathogen contamination in a physical contamination event in an environment subject to contamination,
each treated surface adapted to provide a time-dependent reduction in the number of pathogens available at that treated surface after a physical contamination event,
such that at least a predetermined time after a physical contamination event at a first location on a first treated surface of a first garment, a first physical contact between the first location on the first treated surface and a second location on a second treated surface of a second garment results fewer viable pathogens on the second treated surface compared to an untreated control; and
at least a predetermined time after the first physical contact, a second physical contact between the second location on the second treated surface and a third location on a third treated surface of a third garment results in fewer viable pathogens on the third treated surface compared to an untreated control.

2. The assembly of claim 1, wherein the donnable or drapable garments are selected from gloves, gowns, facemasks, head covers, shoe covers, surgical drape, surgical fenestration or cover, drape, sheets, linens, or padding.

3. The assembly of claim 2, wherein the assembly comprises at least three different donnable or drapable garments.

4. The assembly of claim 2, wherein the garments are medical garments and the environment subject to contamination is a clinical environment.

5. The assembly of claim 1, wherein the reduction in viable pathogens on the third treated surface is at least a 1 log10 CFU reduction of viable pathogens as compared to an untreated control, under ambient conditions.

6. The assembly of claim 1, wherein the reduction in viable pathogens on the third treated surface is at least a 2 log10 CFU reduction of viable pathogens as compared to an untreated control, under ambient conditions.

7. The assembly of claim 5, wherein the 1 log10 CFU reduction of viable pathogens is within about 60 seconds of contact between the second treated surface and the third treated surface.

8. The assembly of claim 6, wherein the 2 log10 CFU reduction of viable pathogens is within about 10 minutes of contact between the second treated surface and the third treated surface.

9. The assembly of claim 1, where in the treated surface is treated with a germicide selected from second biguanide, chlorohexidine, alexidine, and relevant salts thereof, a quaternary ammonium compound, a quaternary siloxane, a polyquaternary amine; metal-containing species and oxides thereof, either in particle form or incorporated into a support matrix or polymer; halogens, a halogen-releasing agent or halogen-containing polymer, a bromo-compound, a chlorine dioxide, a thiazole, a thiocynate, an isothiazolin, a cyanobutane, a dithiocarbamate, a thione, a triclosan, an alkylsulfosuccinate, an alkyl-amino-alkyl glycine, a dialkyl-dimethyl-phosphonium salt, a cetrimide, hydrogen peroxide, 1-alkyl-1,5-diazapentane, cetyl pyridinium chloride, stabilized peroxide, sulfides, bis-phenols, polyphenols, chitosan, anatase TiO2, tourmaline, hydrotropes, chaotropic agents, and synergistic combinations thereof.

10. The according to claim 1, wherein said organic acid includes at least one of the following: acetic, ascorbic, benzoic, citric, glutaric, maleic, polylactic, polyglycolic, propionic, and salicylic acid.

11. The assembly of claim 1 wherein the predetermined time after the physical contamination event is at least 40 seconds.

12. The assembly of claim 11, wherein the predetermined time after the physical contamination event is from about 40 to about 60 seconds.

13. The assembly of claim 1 wherein the predetermined time after the first physical contact is at least 40 seconds.

14. A method of providing a germicidal surface-covering assembly, the method comprising:
providing at least two different donnable or drapable garments, each garment defining at least one treated surface susceptible to pathogen contamination in a physical contamination event in an environment subject to contamination, each treated surface adapted to provide a time-dependent reduction in the number of pathogens available at that treated surface after a physical contamination event;
donning at least one donnable garment on at least one individual in the environment subject to contamination; and
draping at least one drapable garment on at least one surface in the environment subject to contamination; such that:
at least a predetermined time after a physical contamination event at a first location on a first treated surface of a first garment, physical contact between the first location on the first treated surface and a second location on a second treated surface of a second garment results fewer viable pathogens on the second treated surface compared to an untreated control; and
at least a predetermined time after the first physical contact, a second physical contact between the second location on the second treated surface and a third location on a third treated surface of a third garment results in fewer viable pathogens on the third treated surface compared to an untreated control.

15. The method of claim 14, wherein the donnable garments are selected from gloves, gowns, facemasks, head covers, and shoe covers.

16. The method of claim 14, wherein the drapable garments are selected from surgical drapes, surgical covers, sheets, linens, and padding.

17. A method of providing a germicidal surface-covering garment assembly, the method comprising:
providing at least two different donnable garments, each garment defining at least one treated surface susceptible to pathogen contamination in a physical contamination event in an environment subject to contamination, each treated surface adapted to provide a time-dependent reduction in the number of pathogens available at that treated surface after a physical contamination event; and
donning two or more donnable garments on at least one individual in the environment subject to contamination; such that:
at least a predetermined time after a physical contamination event at a first location on a first treated surface of a first garment, physical contact between the first location on the first treated surface and a second location on a second treated surface of a second garment results fewer viable pathogens on the second treated surface compared to an untreated control; and
at least a predetermined time after the first physical contact, a second physical contact between the second location on the second treated surface and a third location on a third treated surface of a third garment results in fewer viable pathogens on the third treated surface compared to an untreated control.

18. The method of claim 17, wherein the garments are selected from gloves, gowns, facemasks, head covers, and shoe covers.
